(19) United States
(12) Patent Application Publication
(10) Pub. No.: US 2010/0210974 A1
(43) Pub. Date: Aug. 19, 2010

(54) AUTOMATIC DISCRIMINATION OF DYNAMIC BEHAVIOUR

(75) Inventors: Peter Nigel Brett, Warwickshire (GB); Xianghong Ma, Warwickshire (GB); Mark T. Elliott, West Midlands (GB)

Correspondence Address: CHRISTIE, PARKER & HALE, LLP, PO BOX 7068, PASADENA, CA, 91109-7068, US

(73) Assignee: ASTON UNIVERSITY, Birmingham (GB)

(21) Appl. No.: 12/664,879

(22) PCT Filed: Jun. 16, 2008
(86) PCT No.: PCT/GB2008/002037

§ 371 Date: Dec. 15, 2009

(30) Foreign Application Priority Data
Jun. 15, 2007 (GB) 0711599.1

(51) Int. Cl. A61B 5/11

(52) U.S. Cl. 600/595

(57) ABSTRACT

There is a distributive sensing technology that can discriminate between the causes of dynamic disturbances. The system applies sensing elements within a sensing medium, and the medium couples the dynamic disturbance and the sensors. By interpreting the responses of the medium, the nature of the disturbance can be discriminated in a way as to determine a description, class or category. There is a method of categorizing the dynamic body behavior, the method comprising: providing a sensing medium during at least a period of the dynamic behavior of the body; providing a plurality of sensors coupled to the sensing medium; obtaining a sensory data time series from each sensor, the sensing medium and the sensors being arranged such that the obtained sensory data time series are not independent from one another; specifying a dynamic behavior category; and processing the sensory data time series. Apparatus for performing the method is also described.

### FIELD OF THE INVENTION

The present invention relates to automatic discrimination of dynamic behaviour. In particular, the invention relates to a method of categorising the dynamic behaviour of a body.

### BACKGROUND OF THE INVENTION

Analysing the dynamic behaviour of a body is important in many areas. For example, consider the area of human gait analysis, i.e. the analysis of a person's manner of walking.

Gait analysis laboratories are essential for the detection of walking disorders in people and for the monitoring of their rehabilitation progress post-operation.

Two tools often used in gait analysis are the use of video based kinematic data, and the use of kinetic force plate data. There is currently a reliance on motion capture systems (i.e. video based kinematic data) to analyse gait rather than platform based devices (i.e. force plates).

Motion capture systems are expensive and rely on a skilled operator to capture the data, so such systems are not always accessible to gait analysis laboratories.

Force plates, on the other hand, can provide very accurate measurements of the force and moment components generated as a person walks over a surface and hence can be used as a tool for diagnosing walking disorders as well as changes in walking over time. However, a standard force plate has two key limitations which, if resolved, would greatly improve the analysis procedure used in gait analysis laboratories.

The first limitation is in the physical dimensions of a force plate. Usually around 0.25 m², their dimensions and structural operation means they can only capture one foot strike. Therefore, for most walking applications a plurality of force plates must be used. To reduce noise, most force plates need to be embedded or secured to the floor they are mounted on. This leads to the issue of how the plates are positioned relative to each other. People have different stride lengths, yet the semi-permanent installation and limited plate dimensions mean that only a narrow range of stride lengths can be properly accommodated. In the worst case this can cause the subject to shorten or lengthen their normal stride and result in measurements being taken that are not representative of the subject's natural gait. Further to this, due to the small dimensions, it has been observed that some patients aim and strike the plate abnormally hard, also creating false readings.

The second limitation of force plates, however, is that they only produce raw measurement data: there is no intelligence in the system to aid with the analysis. A laboratory analyst would have to compile and examine six sets of time series data (three dimensions of forces and moments) for each data capture in order to make a diagnosis. The extra time taken to analyse the data results in less time available for patients to use the gait analysis laboratory and hence increased patient waiting times.

### SUMMARY OF THE INVENTION

There is described a distributive sensing technology that is able to discriminate between the causes of dynamic disturbances. The system applies a limited number of sensing elements within a sensing medium. The sensing medium itself provides a nonlinear coupling between the dynamic disturbance and the sensors. By interpreting the simultaneous, collective sensed responses of the medium, the nature of the disturbance can be discriminated in such a way as to determine a description, class or category. As an example, a continuous surface sensing medium is described that is able to output categories of the behaviour and motion of human or animal subjects moving over the surface. Since the sensing surface is a continuous medium, a response from a sensing element can be obtained for a disturbance applied over a large proportion of the surface medium.

There is an integrated process to interpret the simultaneous response transients. This can be in the time, wavelet and/or frequency domains, and may include the identification of features in the sensory transients. An inference scheme is used to discriminate the nature of the dynamic disturbance. Nonlinearity of the coupling functions between sensory outputs enhances the ability of the system to discriminate between types of disturbances. This could relate to types of behavioural disturbances in terms of balance, ambulation or sporting technique, for example. Interpretation of behaviour based on either intermittent or continuous contact of a dynamic body with the medium is possible. The sensing medium or sensing surface enables totally unconstrained motion of the subject, and is able to interpret slipping and multiple points of disturbances such as people using walking aids. Sizing, position and orientation of contacting objects are also possible to determine, as are parameters relating to height, posture, build and respiration of a person or animal.

The performance of the system is dependent on the response of the medium, and sensitivity of sensing output to the disturbances. For some applications, it is necessary to place some sensing elements closer to the disturbance point on the medium, should attenuation of the response to the disturbance occur as a result of the impedance of the medium. In the example case, when detecting the person on a surface, it can be important to deploy sensing elements to the person or to a tool or equipment they maybe handling or manipulating in the activity to be monitored. In such cases, the sensing medium would extend to the structure of the surface, the person and/or the tool. So the sensing medium is defined by the nature of the application. Sensors applied on tools or equipment held by the subjects would enable the detection of the disturbance input at source, and other subtle disturbances that may affect the triggering or timing of a sequential activity of the impact on the disturbance itself.

When the sensing medium is a continuous sensing surface, the surface may be vibrated dynamically with one or more actuated sources in order to detect objects, contact and other metrics. The location of the sources is dependent on the application. The size or shape of the sensing surface is not prescriptive. To date, example systems have been deployed successfully over a scale ranging from a few tens of microns up to a few metres. The miniature size is only dependent on the limits of manufacturing capability. Vibration of the sensing surface is particularly advantageous in cases where a contacting object does not impose a significant force on the sensing surface. In such cases, it may be difficult to detect the signals in the field of strain in the sensing surface which enable the dynamic behaviour of the body to be categorised. Thus, the sensing surface is vibrated towards and away from the body so as to produce a stronger signal in the field of strain in the sensing surface due to the presence of the body. As an example, a vibrating sensing surface with a typical dimension of 0.1 mm across may be used to detect contact and discriminate cells and cell structures. Thus, the system may be used for the discrimination of mechanical characteristics and behaviour.

The body itself may be a person or an animal, or may alternatively be an inanimate object in dynamic motion, such as a pendulum or a bouncing ball. The body could also be a dynamic device operated by a person (i.e. the body whose dynamic behaviour is being categorised includes both the person and the device). Examples are people using gymnasium equipment or other therapeutic equipment, such as in physiotherapy, or people using wheelchairs or other vehicles. In such cases, the sensing medium is used to categorise how the person is performing in their use of the device. Thus, the style of motion or the performance is determined so as to judge correct use of the device or competency, or in order to train the individual. In one example, the device and the person operating it are placed onto a deformable sensing surface together. For wheelchairs, such a system could be used to either check the set-up of a wheelchair for a user, or to train a user in the optimum way to move in order to propel the wheelchair (e.g. optimum seated position and optimum movement of the arms). Rather than the use of a sole sensing surface beneath the device and user, at least a part of the sensing medium could alternatively/additionally be integrated into the wheelchair itself. For gymnasium equipment such as running machines, exercise bicycles and lifting machines, or even weights and exercise mats, a sensing surface could be applied by merely placing the equipment and the user onto the surface to discriminate motion and performance of the individual. The sensing medium could alternatively be incorporated into the machine. Thus, when the body in dynamic motion is a person operating a dynamic device, possible applications include monitoring or screening patients, evaluating metrics, physiological processes and recognition.

According to a first aspect of the present invention, there is provided a method of categorising the dynamic behaviour of a body, the method comprising: providing a sensing medium coupled to the body during at least a period of the dynamic behaviour of the body; providing a plurality of mutually spaced sensors coupled to the sensing medium; obtaining a respective sensory data time series from each sensor during the dynamic behaviour of the body, the sensing medium and the sensors being arranged such that the obtained sensory data time series are not independent from one another; specifying a dynamic behaviour category; and processing the sensory data time series so as to determine whether the dynamic behaviour of the body is in the specified dynamic behaviour category.

Thus, there is described a distributive sensing technology that is able to discriminate between the causes of disturbances automatically. The distributive approach to tactile sensing utilises the response of a continuous sensing medium that is monitored at multiple points from which the nature of the contact over a large proportion of the medium can be inferred. Furthermore, the sensing medium provides a nonlinear coupling between the dynamic behaviour of the body and the sensor outputs such that the sensors indirectly sense the dynamic behaviour of the body via the sensing medium. A small number of discrete sensors are provided that respond in a non-independent (i.e. coupled) manner. Various types of sensors are envisaged within the scope of the invention. The positioning of the sensors on the sensing medium should also be varied depending on the dynamic behaviour category of interest. An interpreting algorithm is used to interpret the sensor outputs. The body may be a person or an animal, or may alternatively be an inanimate object in dynamic motion, such as a pendulum or a bouncing ball. Optionally, the sensing medium is coupled to the body intermittently during the dynamic behaviour of the body. For example, the intermittent contact of a person running on a deformable sensing surface. Alternatively, the sensing medium is coupled to the body continuously during the dynamic behaviour of the body. For example, analysis of a person's golf swing while standing on a deformable sensing surface.

Optionally, during the processing step, the sensory data time series from each sensor is processed together with the sensory data time series from each of the other sensors. Thus, the sensory data time series are processed collectively rather than processing the sensory data time series from each sensor separately or individually. This aids with the analysis of nonlinearity systems.

Optionally, the processing step comprises normalising one or more of the amplitude and duration of each sensory data time series.

Optionally, the processing step comprises smoothing each sensory data time series. This reduces noise and allows the study of lower frequency variations only.

Optionally, the processing step comprises differentiating each sensory data time series with respect to time to create a respective derivative sensory data time series. In this way, the analysis of the dynamic behaviour of the body is based at least partially on the rate of change measured at each sensor. This is important in some applications. Optionally, the processing step comprises smoothing each derivative sensory data time series.

Optionally, the processing is nonlinear. This gives better results when there are nonlinear relationships between the dynamic behaviour of the body and the sensor outputs as well as between the outputs of each sensor.

Optionally, the processing step comprises calculating representative sensory data time series for the specified dynamic behaviour category at each sensor. This step thereby gives some form of comparison with the sensory data time series obtained during the dynamic behaviour to be analysed. The representative data time series may be analysed for each sensor output, or for multiple sensor outputs, or the coupling transients to be used to automatically interpret a dynamic behaviour category. Optionally, the processing step further comprises calculating a cost value to represent the difference between the sensory data time series and the representative sensory data time series. Optionally, the representative sensory data time series are calculated using training data, wherein it is known a priori whether the dynamic behaviour of the training data is in the specified dynamic behaviour category. Optionally, calculating the representative sensory data time series comprises calculating mean sensory data time series for all training data in the specified dynamic category. Optionally, calculating the representative sensory data time series comprises calculating mean sensory data time series for all training data not in the specified dynamic category. Optionally, calculating the representative sensory data time series comprises calculating sensory data standard deviation time series for all training data in the specified dynamic category. Optionally, calculating the representative sensory data time series comprises calculating sensory data standard deviation time series for all training data not in the specified dynamic category.

Optionally, the processing step comprises use of a neural network having inputs and an output, the inputs being responsive to the sensory data time series, the output being a determination of whether the dynamic behaviour of the body is in the specified dynamic behaviour category. Optionally, the neural network inputs are cost values corresponding to each sensor.

Optionally, the processing step comprises analysing the sensory data time series in one or more of the time domain, the frequency domain and the wavelet domain.

Optionally, the sensing medium comprises at least a portion of the body.

Optionally, the body is a person or animal and the sensing medium comprises a tool or equipment held by the person or animal or a device used by the person or animal. In this embodiment, sensors are applied on the tools or equipment or device to enable the detection of disturbance input at source, as well as other subtle disturbances that may affect the triggering or timing of a sequential activity of the impact on the disturbance itself.

Optionally, the method further comprises inferring a state of the body based on the determination of whether the dynamic behaviour of the body is in the specified dynamic behaviour category. For example, a state of balance of the body could be inferred.

Optionally, the method further comprises inferring a cause of the dynamic behaviour of the body based on the determination of whether the dynamic behaviour of the body is in the specified dynamic behaviour category.

Optionally, the body is a person or animal and the method further comprises inferring a medical condition of the person or animal based on the determination of whether the dynamic behaviour of the person or animal is in the specified dynamic behaviour category.

Optionally, the body is a person and the method further comprises inferring performance of sporting activities by the person or sporting technique of the person based on the determination of whether the dynamic behaviour of the person is in the specified dynamic behaviour category.

Optionally, the sensors are arranged to sense one or more of strain, deformation, velocity and deflection of the sensing medium. Other physical and dynamic properties may also be sensed.

Optionally, the method further comprises inferring one or more of the size, position and orientation of the body based on the determination of whether the dynamic behaviour of the body is in the specified dynamic behaviour category.

Optionally, the body is a person or animal and the method further comprises inferring one or more of the height, weight, posture, build and respiration of the person or animal based on the determination of whether the dynamic behaviour of the person or animal is in the specified dynamic behaviour category.

Optionally, the processing step comprises identifying transient features in the sensory data time series.

Optionally, the sensing medium is a deformable sensing surface. In this case, the sensors are arranged to sense the dynamics of the surface (e.g. the deformation of the surface).

Advantageously, the method further comprises the steps of: providing an actuator arranged to move the sensing surface; and moving the sensing surface with the actuator during at least a period of the dynamic behaviour of the body. More advantageously, the moving step comprises vibrating the sensing surface with the actuator in a direction towards and away from the body during at least a period of the dynamic behaviour of the body.

Optionally, the body is a person, the dynamic behaviour is walking, and the deformable sensing surface is arranged to receive at least two footsteps during the normal walking of an adult male. Previously, the distributive sensing method has only been used in small scale, static applications. However, in these embodiments, the method is applied to a large platform area which is able to capture a single human gait stride. Thus, a surface medium is described that is able to output description of the behaviour and motion of human subjects moving over the surface.

In the embodiment where the dynamic behaviour is walking, the behaviour category could be a pre-defined gait category. For example, the gait category could be “wearing a rucksack”, “limping”, “tiptoeing”, “shuffling”, etc. Alternatively, the dynamic behaviour could be running or jogging, etc.

Optionally, the sensing medium is non-planar. It could be in the form of a step, a bed or a seat, for example.

According to a second aspect of the present invention, there is provided apparatus for performing the method of the first aspect.

### BRIEF DESCRIPTION OF THE DRAWINGS

Embodiments of the present invention will now be described by way of example with reference to the accompanying drawings in which:

FIG. 1 shows a sensing platform in accordance with one embodiment of the invention;

FIG. 2 schematically illustrates anticipate footstep locations on an elongate platform including a gait initiation platform, the sensing platform of FIG. 1, and a gait termination platform; and

FIGS. 3a, 3b and 3c show representative sensory data time series for three sensors coupled to the sensing platform when a subject wearing a backpack walks across the sensing platform.

### DETAILED DESCRIPTION OF A PREFERRED EMBODIMENT

A method of analysing the dynamic behaviour of a body is described. In this embodiment, the body is a person, and the dynamic behaviour is walking. A sensing medium in the form of a sensing platform with a deformable sensing plate is used. The output of deflection sensors is then processed using a neural network to analyse the difference between a person's normal gait when walking on the sensing plate as compared to their gait when carrying a 10 kg mass in a back pack. However, it will be understood that this embodiment is by way of example only and other embodiments are envisaged within the scope of the claims.

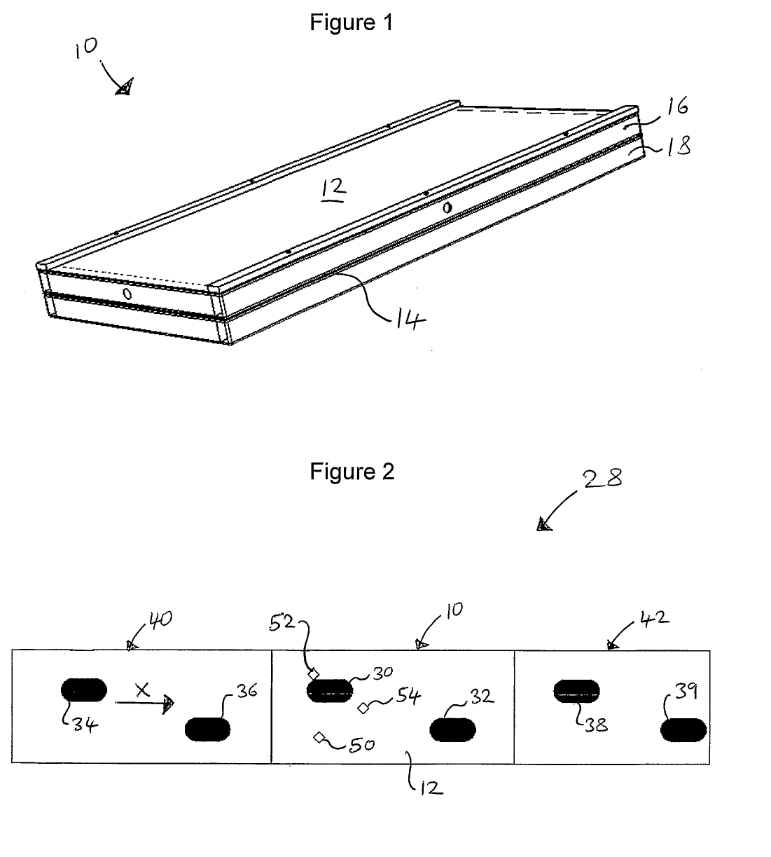

FIG. 1 shows the sensing platform 10. The sensing platform 10 has a flexible sensing plate 12 that is able to deform to enable the sensing of the gait pattern generated by a subject standing on the plate. The sensing plate 10 is therefore different to a force plate which is required to have a high stiffness in order to operate. The sensing platform 10 and sensing plate 12 are both generally elongate and rectangular in shape.

The sensing platform 10 further comprises an elongate rectangular base plate 14 on which to mount sensors (described later) and the sensing plate 12. The base plate 14 is sandwiched between an upper frame 16 and a lower frame 18 constructed from rectangular steel tubing.

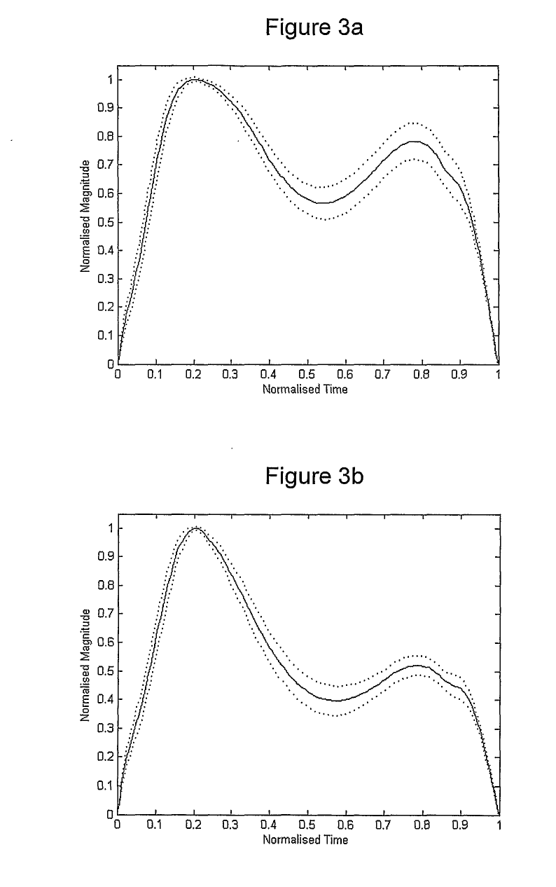

The sensing platform 10 has a length of 150 cm and a width of 70 cm such that it is able to record two footsteps (i.e. one left footstep 30 and one right footstep 32) as a subject walks over the sensing platform 10 in a direction X, as shown in FIG. 2.

FIG. 2 shows a walkway 28 comprising the sensing platform 10 and two additional platforms placed before and after the sensing platform 10. The first additional platform is a gait initiation platform 40 and the second additional platform is a gait termination platform 42. The platforms 40, 42 are simple wooden constructions with adjustable feet to enable them to sit flush and level with the sensing platform 10, eliminating any trip hazards. In this embodiment, a relatively short, two-step gait initiation is used. This involves two footsteps (i.e. a left footstep 34 and a right footstep 36) being taken on the gait initiation platform 40 by a subject prior to measurements being taken during the two footsteps 30, 32 on the sensing platform 10. Two further footsteps 38, 39 are then taken on the gait termination platform 42.

In use, the deflection of the sensing plate 12 caused by each footstep 30, 32 is measured using three sensors 50, 52, 54. In this embodiment, the sensors 50, 52, 54 are non-contact, optical deflection sensors positioned on the base plate 14 under the sensing plate 12. Each sensor 50, 52, 54 comprises an infrared LED and integrated phototransistor. The LED output reflects off a lower surface of the sensing plate 12 onto the phototransistor. The sensors 50, 52, 54 are located in a triangular formation as shown in FIG. 2 such that deflection differences both along the walkway 28 and across the walkway 28 could be detected.

To show the ability of the smart sensing platform to discriminate different gait patterns, an experiment is described to detect and classify a small change in a single person's gait. In this experiment the subject is required to walk along the walkway 28 with and without a backpack style bag containing a 10 kg mass, representing abnormal and normal walking respectively. The 10 kg mass is intended to create a small but detectable change in the subject's gait due to changes in their stance and balance. Having identified the “with backpack” gait category/class as the one to be targeted in this example, the experimental method described below enables determination of whether or not a particular capture of a person walking falls into the “with backpack” class. Note that the “with backpack” class will hereinafter be referred to as the Backpack class. In addition, the alternative “without backpack” class will hereinafter be referred to as the Natural class. Each set of measurements of a subject walking is termed a “capture”.

Initially, to enable classification of a given capture, a representative Backpack class template is generated. Using previously captured data as a training set, the template is generated based on fifty captures for the Backpack class. A separate template is created for each of the three sensors 50, 52, 54. Each training capture is filtered using a moving average filter to remove noise and smooth the curve. The amplitude is then normalised between zero and one. Each capture has to be of equal length and hence is mapped on to an m=200 element vector over the time interval [0,1]. Any captures found to be less than two hundred samples long are discarded. Given n training captures, the resulting dataset is three sets of n×m matrices. Based on the n training captures, the mean of each of the 200 elements was calculated to produce a template vector for each sensor 50, 52, 54:

$$\begin{matrix}\overline{x} = \{ {\overline{x}}_{1} , {\overline{x}}_{2} , … {\overline{x}}_{k} , … , {\overline{x}}_{m} \} & ( 1 ) \\ \text{where:} {\overline{x}}_{k} = \frac{1}{n} {\sum }_{i = 1}^{n} {t}_{i , k} & ( 2 )\end{matrix}$$

with the i^{th} training capture vector defined as Tᵢ={t_{i,1},t_{i,2}, . . . t_{i,k}, . . . , t_{i,m}} for i=1,2, . . . , n.

Similarly, a vector of standard deviations is also calculated:

\hat{σ}={σ₁,σ₂, . . . σₖ, . . . , σₘ} (3)

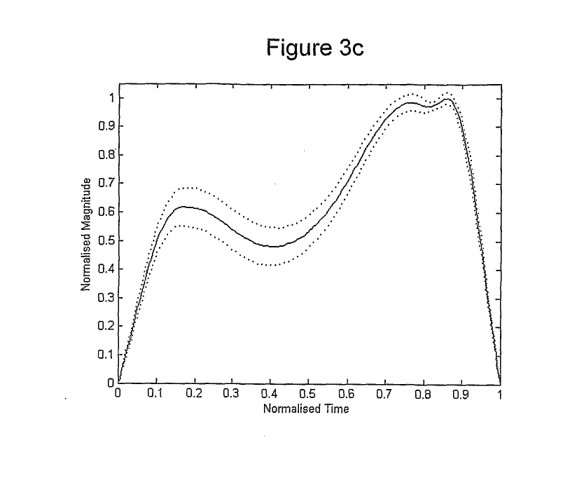

A mean vector x̅ is generated for each of the three sensors 50, 52, 54 and used as the template. In addition to this, the standard deviation (s.d) vector \hat{σ} is added and subtracted from the mean vector x̅ to produce a “soft” boundary above and below the template curve, as shown in FIGS. 3a to 3c. FIG. 3a shows a typical Backpack template for the first sensor 50, FIG. 3b shows a typical Backpack template for the second sensor 52, and FIG. 3c shows a typical Backpack template for the third sensor 54.

The captured data used for testing, R={r₁,r₂, . . . rₖ, . . . , rₘ}, is processed the same way as the template data Tᵢ={t_{i,1},t_{i,2}, . . . t_{i,k}, . . . , t_{i,m}}. The absolute distance dₖ between each sample rₖ in the test data vector and the corresponding sample tₖ in the template vector is calculated and divided by the standard deviation σₖ for that sample:

$${d}_{k} = \frac{\vert {r}_{k} - {\overline{x}}_{k} \vert}{{σ}_{k}} k = 1 , 2 , … , m \qquad ( 4 )$$

By dividing by the standard deviation, the variation in a person's walking style is taken in to account, hence weighting the distance score to that template appropriately. Each comparison of the test data to a template produces a cost value C^{R}, defined as the mean value of the distances defined in equation (4) as follows:

$${C}^{R} = \frac{1}{m} {\sum }_{k = 1}^{m} {d}_{k} \qquad ( 5 )$$

In addition, the captured data and the mean vector are both differentiated. The two resulting gradient curves are compared in the same way as the time based data as shown in equation (4). In this way, a gradient cost value C^{dR} is defined as:

$${C}^{dR} = \frac{1}{m - 1} {\sum }_{k = 1}^{m - 1} \frac{\vert δ {r}_{k} - δ {\overline{x}}_{k} \vert}{{δσ}_{k}} \qquad ( 6 )$$

Finally, the resulting overall cost C is calculated as:

C=C^{R}·C^{dR} (7)

The cost C is calculated for each of the three sensors 50, 52, 54 for the Backpack class

In order to determine whether or not a given test capture falls into the Backpack category, the system requires some form of threshold level defining. If the distance scores from the three sensors 50, 52, 54 were found to be less than the threshold, then they would be assigned to the Backpack class, otherwise they would be assigned to the alternative Natural class. Since the threshold boundary between the classes is likely to be nonlinear in this case, a neural network is introduced to the system. Use of a neural network provides a number of advantages as described below.

Firstly, the use of a neural network enables the definition of a nonlinear boundary between the two classes. For this experiment, it was likely that the relationship between the sensors and the distance thresholds between the two classes was nonlinear. Neural networks are able to define nonlinear relationships between their inputs and outputs and therefore provided a better a classification accuracy than using a linear method.

Secondly, the use of a neural network allows coupling of the data from each of the three sensors 50, 52, 54. Rather than investigate the sensor outputs individually, the distributive sensing method is used in this experiment to consider the three sensor outputs together. Using a neural network enables the system to identify complex coupled relationships between the sensors 50, 52, 54 and relate them to the Backpack class.

Thirdly, the use of a neural network allows the dependency on where the footstep 30 occurs to be removed. One of the nonlinear relationships mentioned above is due to the position of the footstep 30 on the sensing plate 12. The neural network therefore identifies how the cost values from the three sensor outputs change with respect to each other when the footstep 30 landed in different places.

A network architecture comprising a feedforward multi-layer perceptron (MLP) is used in this embodiment. The neural network has an input layer with three input nodes, a hidden layer with two nodes, and an output layer with a single output node. The costs C from each sensor 50, 52, 54 are used as the three inputs. A log-sigmoid transfer function is used both on the hidden layer and output layer nodes. A Bayesian Regularisation training method is used. Using randomised start weights, the neural network is repeatedly trained until a well balanced classifier is established.

It takes approximately three seconds for the system described herein to identify the predicted class (Backpack or Natural) after a subject has walked over the sensing platform 10. Because of the two possible classifications, binary classifier notation is introduced and used to assess the performance of this system. Table 1 identifies the two possible classes as positive and negative events and relates these to the possible outcome of this experiment. The notation of positive and negative events derives from the common usage of these measurements in medical trials. In this experiment, the aim is to identify when the person is wearing the backpack. Therefore, for notation's sake, the event where the person is wearing the backpack will be identified as positive, whilst the negative event is defined as the person walking naturally. So for example, a True Negative event occurs when the classifier correctly identifies the person walking naturally.

TABLE 1

| Outcome | Label | Event |
|---|---|---|
| True | TP | Backpack class correctly identified |
| Positive | | |
| True | TN | Natural class correctly identified |
| Negative | | |
| False | FP | Natural class incorrectly identified as |
| Positive | | Backpack class |
| False | FN | Backpack class incorrectly identified as |
| Negative | | Natural class |

The results from the optimised neural network after 46 live trials are shown in the Table 2. Of the 46 live trials, in reality, 23 comprised natural walking and 23 comprised walking with a backpack.

TABLE 2

Predicted class

| | | Natural | Backpack |
|---|---|---|---|
| Actual class | Natural | 20 | 3 |
| | Backpack | 2 | 21 |

Using Table 2, it is possible to calculate the specificity and sensitivity of the classifier. Specificity is a measure of how accurate the classifier is at identifying a True Negative result and is defined as:

$$Specificity = \frac{TN}{TN + FP} \qquad ( 8 )$$

Similarly, sensitivity is a measure of how accurate the classifier is at identifying a True Positive result and is defined as:

$$Sensitivity = \frac{TP}{TP + FN} \qquad ( 9 )$$

Related measures of accuracy are the Positive Predictive Value (PPV) and Negative Predictive Value (NPV). In this experiment, PPV states the probability of the person wearing the backpack when the system states they are and is defined as PPV=TP/(TP+FP). Similarly, NPV states the probability that the person is walking naturally when the system states that is the case and is defined as NPV=TN/(TN+FN). Note that, unlike sensitivity and specificity, PPV and NPV are affected by the number of vectors present in each class.

The above classification performance factors are shown in Table 3 and show a good level of accuracy. Additionally, both classes are well balanced with the specificity and sensitivity values very close together. Large variations between the two values would indicate the classifier is more biased to choosing a particular class.

TABLE 3

| Accuracy measure | Value |
|---|---|
| Sensitivity | 0.91 |
| Specificity | 0.87 |
| PPV | 0.88 |
| NPV | 0.91 |
| Overall | 89% |

Having optimised the neural network, one of the advantages of this method is that live captures can continue to be stored during use to provide further training data when accompanied with the correct class, leading to an adaptive system that can learn gait patterns over time.

In conclusion, a smart sensing platform for detecting and discriminating small changes in a person's gait has been described. The distance based algorithms rely on the overall shape of the gait pattern and could therefore be applied to any walking disorder that affects the gait pattern. Due to the high accuracy of the method at discriminating between gait categories, this method could be applied to clinical applications, where it could be used to identify certain pathological or neurological gait disorders.

As an example of a clinical application, cerebral palsy classification is investigated by many publications as indicated by the review undertaken by Dobson et al. entitled “Gait classification in children with cerebral palsy: A systematic review” (Gait and Posture 25, 2007, 140-152). Dobson et al. state that this area is of interest due to the “diversity of gait deviations observed in children with cerebral palsy” and that “gait classifications may enable clinicians to differentiate gait patterns into clinically significant categories that assist with clinical decision-making”.

Although preferred embodiments of the invention have been described, it is to be understood that these are by way of example only and that various modifications may be contemplated.

In alternative embodiments using neural networks, the neural network architecture, the number of hidden nodes, and the neural network training method could be varied. For example, Radial Basis Function networks could be used to define the network architecture. Also, Early Stopping training methods could be used instead of Bayesian Regularisation training methods.

Alternatively, a processing algorithm other than a neural network may be used.

1. A method of categorizing a dynamic behavior of a body, the method comprising:
providing a sensing medium coupled to the body during at least a period of the dynamic behavior of the body;
providing a plurality of mutually spaced sensors coupled to the sensing medium;
obtaining a respective sensory data time series from each sensor during the dynamic behavior of the body, the sensing medium and the sensors being arranged such that the obtained sensory data time series are not independent from one another;
specifying a dynamic behavior category; and
processing the sensory data time series so as to determine whether the dynamic behavior of the body is in the specified dynamic behavior category.

2. The method of claim 1 wherein the sensing medium is coupled to the body intermittently during the dynamic behavior of the body.

3. The method of claim 1 wherein the sensing medium is coupled to the body continuously during the dynamic behavior of the body.

4. The method of claim 1 wherein, during the processing step, the sensory data time series from each sensor is processed together with the sensory data time series from each of the other sensors.

5. The method of claim 1 wherein the processing step comprises differentiating each sensory data time series with respect to time to create a respective derivative sensory data time series.

6. The method of claim 1 wherein the processing is nonlinear.

7. The method of claim 1 wherein the processing step comprises analyzing representative sensory data time series for the specified dynamic behavior category at each sensor.

8. The method of claim 7 wherein the processing step further comprises calculating a cost value to represent the difference between the sensory data time series and the representative sensory data time series.

9. The method of claim 7 wherein the representative sensory data time series are calculated using training data, wherein it is known a priori whether the dynamic behavior of the training data is in the specified dynamic behavior category.

10. The method of claim 1 wherein the processing step comprises applying a neural network having inputs and an output, the inputs being responsive to the sensory data time series, the output being a determination of whether the dynamic behavior of the body is in the specified dynamic behavior category.

11. The method of claim 10 wherein the processing step further comprises calculating a cost value to represent the difference between the sensory data time series and the representative sensory data time series and wherein the neural network inputs are cost values corresponding to each sensor.

12. The method of claim 1 wherein the processing step comprises analyzing the sensory data time series in one or more of the time domain, the frequency domain and the wavelet domain.

13. The method of claim 1 wherein the sensing medium comprises at least a portion of the body.

14. The method of claim 1 wherein the body is a person or animal and the sensing medium comprises a tool or equipment held by the person or animal or a device used by the person or animal.

15. The method of claim 1 further comprising inferring a state of the body based on the determination of whether the dynamic behavior of the body is in the specified dynamic behavior category.

16. The method of claim 1 further comprising inferring a cause of the dynamic behavior of the body based on the determination of whether the dynamic behavior of the body is in the specified dynamic behavior category.

17. The method of claim 1 wherein the body is a person or animal and the method further comprises inferring a medical condition of the person or animal based on the determination of whether the dynamic behavior of the person or animal is in the specified dynamic behavior category.

18. The method of claim 1 wherein the body is a person and the method further comprises inferring performance of sporting activities by the person or sporting technique of the person based on the determination of whether the dynamic behavior of the person is in the specified dynamic behavior category.

19. The method of claim 1 wherein the sensors are arranged to sense one or more of strain, deformation, deflection and velocity of the sensing medium.

20. The method or claim 1 further comprising inferring one or more of the size, position and orientation of the body based on the determination of whether the dynamic behavior of the body is in the specified dynamic behavior category.

21. The method of claim 1 wherein the body is a person or animal and the method further comprises inferring one or more of the height, weight, posture, build and respiration of the person or animal based on the determination of whether the dynamic behavior of the person or animal is in the specified dynamic behavior category.

22. The method of claim 1 wherein the processing step comprises identifying transient features in the sensory data time series.

23. The method of claim 1 wherein the sensing medium is a deformable sensing surface.

24. The method of claim 23 further comprising the steps of:
providing an actuator arranged to move the sensing surface; and
moving the sensing surface with the actuator during at least a period of the dynamic behavior of the body.

25. The method of claim 24 wherein the moving step comprises vibrating the sensing surface with the actuator in a direction towards and away from the body during at least a period of the dynamic behavior of the body.

26. The method of claim 23 wherein the body is a person, the dynamic behavior is walking, and the deformable sensing surface is arranged to receive at least two footsteps during the normal walking of an adult male.

27. The method of claim 1 wherein the sensing medium is non-planar.

28. The method of claim 1 wherein the specifying step comprises specifying a plurality of dynamic behavior categories, and wherein the processing step comprises processing the sensory data time series so as to discriminate the dynamic behavior of the body based on the specified dynamic behavior categories.

29. Apparatus for performing the method of claim 1.
